(19) United States
(12) Patent Application Publication
(10) Pub. No.: US 2015/0045437 A1
(43) Pub. Date: Feb. 12, 2015

(54) TRANSDERMAL DELIVERY KITS

(71) Applicant: ASCLEMED USA, INC. dba EnovaChem Manufacturing, Torrance, CA (US)

(72) Inventor: Robert Prentice Nickell, Torrance, CA (US)

(21) Appl. No.: 14/455,658

(22) Filed: Aug. 8, 2014

Related U.S. Application Data
(60) Provisional application No. 61/863,825, filed on Aug. 8, 2013.
(60) Provisional application No. 62/001,879, filed on May 22, 2014.

(51) Int. Cl. A61J 3/00; A61K 31/197; A61J 1/20; A61K 31/167; A61K 31/135; A61J 1/14; A61K 31/137; A61K 31/192

(52) U.S. Cl. CPC A61J 3/00; A61K 31/137; A61K 31/197; A61K 31/192; A61K 31/167; A61K 31/135; A61J 1/1412; A61J 1/20
USPC 514/567; 514/654; 514/570; 514/626; 514/646; 206/568; 206/570; 514/569

(57) ABSTRACT

Provided are kits containing a base composition and an active pharmaceutical ingredient for transdermal delivery of active pharmaceutical ingredients. Examples of active pharmaceutical ingredients include amitriptyline, baclofen, cyclobenzaprine HCl, ibuprofen, lidocaine HCl, naproxen and tramadol, ketoprofen, diclofenac, phenylbutazone, mefenamic acid, flubiprofen, piroxicam, guaifenasin, prilocaine, bupivicaine, tetracaine, nifedipine, verapamil, orphenadrine, imipramine, ketamine, gabapentin, carbamazepine, menthol, capsaicin, clonidine, dexamethasone, dextromethorphan, testosterone, progesterone, and estrogens.

### CROSS-REFERENCE

The present application can claim the benefit of U.S. Appl. No. 62/001,879, filed on May 22, 2014, and U.S. Appl. No. 61/863,825, filed on Aug. 8, 2013, both of which is incorporated herein by reference in its entirety.

### BACKGROUND SECTION OF THE INVENTION

Drugs can be administered to a patient through various routes, such as transdermal delivery. Currently medical practitioners have limited availability of drugs in a suitable form for transdermal delivery. In addition, pre-formulated drugs are prone to degradation and do not allow a medical practitioner to make a desired formulation. There is a need in the prior art for kits that address the above-mentioned shortcomings.

### SUMMARY SECTION OF THE INVENTION

Provided is a kit comprising or consisting or consisting essentially of: a) a first container of an active pharmaceutical ingredient (API); b) a second container of a base composition suitable for transdermal delivery for mixing with the API; c) a third container, wherein the first and the second container are placed inside a third container. The composition can be in the form of an ointment, a cream, a rinse, or a gel. The base composition can comprise phospholipids. The API can be selected from the group consisting of: amitriptyline, baclofen, cyclobenzaprine HCl, ibuprofen, lidocaine HCl, naproxen, and tramadol. The API can be selected from the group consisting of: ketoprofen, diclofenac, phenylbutazone, mefenamic acid, flubiprofen, piroxicam, guaifenasin, prilocaine, bupivicaine, tetracaine, nifedipine, verapamil, orphenadrine, imipramine, ketamine, gabapentin, carbamazepine, menthol, capsaicin, clonidine, dexamethasone, dextromethorphan, testosterone, progesterone, and estrogens. The kit can further comprise a stirrer placed inside the third container, which is typically a box. The kit can further comprise an instruction sheet placed inside the third container. The third container can be as box with a lid. The API can be amitriptyline. The API can be baclofen. The API can be cyclobenzaprine HCl. The API can be ibuprofen. The API can be lidocaine HCl. The API can be naproxen. The API can be tramadol

The kit can comprise, consist essentially of, or consist of: a) 2-260 g total weight of the base composition and the API combined; b) 1-20 grams of API, with the API having a strength of 1-20% USP (United States Pharmacopeia); and c) 1-240 grams of the base composition. The kit can comprises, consist essentially of, or consist of: a) 41-140 g total weight of the base and the API combined; b) 1-20 grams of API, with the API having a strength of 1-20% USP; and c) 40-120 grams of the base. The weight of the API and the base composition can be in the range of about 40 grams to about 140 grams. The weight of the API and the base composition can be in the range of about 60 grams to about 120 grams. The weight of the API can be 1 gram to about 20 grams. The strength of the API can be about 1% to about 10% USP. The weight of the API and the base composition base in the range of about 40 grams to about 140 grams. API can be 1 gram to about 20 grams, with the rest being weight of the base composition. The API can be amitriptyline, weight of the API and the base composition can be about 60 grams, with the weight of the API being 1.2 grams, and wherein the API has strength of 2% USP. The API can be amitriptyline, weight of the API and the base composition can be about 120 grams, with the weight of the API being 2.4 grams, and wherein the API has strength of 2% USP. The API can be baclofen, weight of the API and the base composition can be about 60 grams, with the weight of the API being 0.6 grams, and wherein the API has strength of 1% USP. The API can be baclofen, weight of the API and the base composition can be about 120 grams, with the weight of the API being 1.2 grams, and wherein the API has strength of 1% USP. The API can be cyclobenzaprine HCl, weight of the API and the base composition can be about 120 grams, with the weight of the API being 2.4 grams, and wherein the API has strength of 2% USP. The API can be ibuprofen, weight of the API and the base composition can be about 120 grams, with the weight of the API being 12 grams, and wherein the API has strength of 10% USP. The API can be lidocaine HCl, weight of the API and the base composition can be about 60 grams, with the weight of the API being 6 grams, and wherein the API has strength of 10% USP. The API can be lidocaine HCl, weight of the API and the base composition can be about 60 grams, with the weight of the API being 3 grams, and wherein the API has strength of 5% USP. The API can be lidocaine HCl, weight of the API and the base composition can be about 120 grams, with the weight of the API being 12 grams, and wherein the API has strength of 10% USP. The API can be lidocaine HCl, weight of the API and the base composition can be about 120 grams, with the weight of the API being 6 grams, and wherein the API has strength of 5% USP. The API can be naproxen, weight of the API and the base composition can be about 60 grams, with the weight of the API being 6 grams, and wherein the API has strength of 10% USP. The API can be naproxen, weight of the API and the base composition can be about 120 grams, with the weight of the API being 12 grams, and wherein the API has strength of 10% USP. The API can be tramadol weight of the API and the base composition can be about 60 grams, with the weight of the API being 3 grams, and wherein the API has strength of 5% USP. The API can be tramadol, weight of the API and the base composition can be about 120 grams, with the weight of the API being 6 grams, and wherein the API has strength of 5% USP.

Provided is a method of transdermally administering an Active Pharmaceutical Ingredient (API) comprising: a) removing a first container of a base composition from a third container; b) removing a second container of an active pharmaceutical ingredient (API) from the third container; c) opening the first container; d) opening the second container; e) mixing the active pharmaceutical ingredient (API) and the base composition to obtain a pharmaceutical composition for transdermal delivery; f) applying the pharmaceutical composition to skin.

### BRIEF DESCRIPTION OF THE DRAWINGS

FIG. 1 illustrates a box with a lid where each component of the kit is placed.

FIG. 2 illustrates an instruction sheet.

FIG. 3 illustrates a container of a cream (base composition).

FIG. 4 illustrates a container of an active pharmaceutical ingredient.

FIG. 5 illustrates a stirrer.

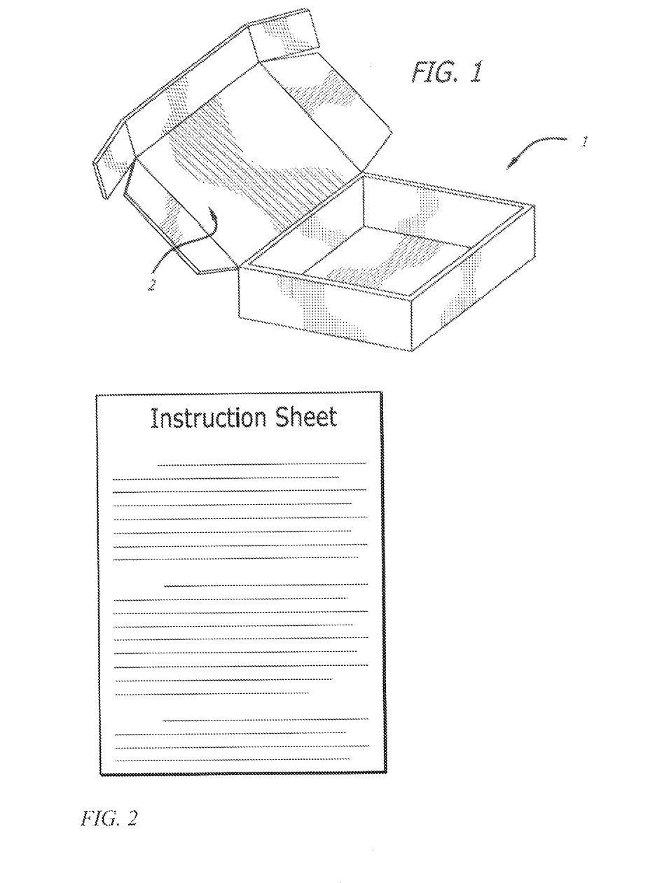

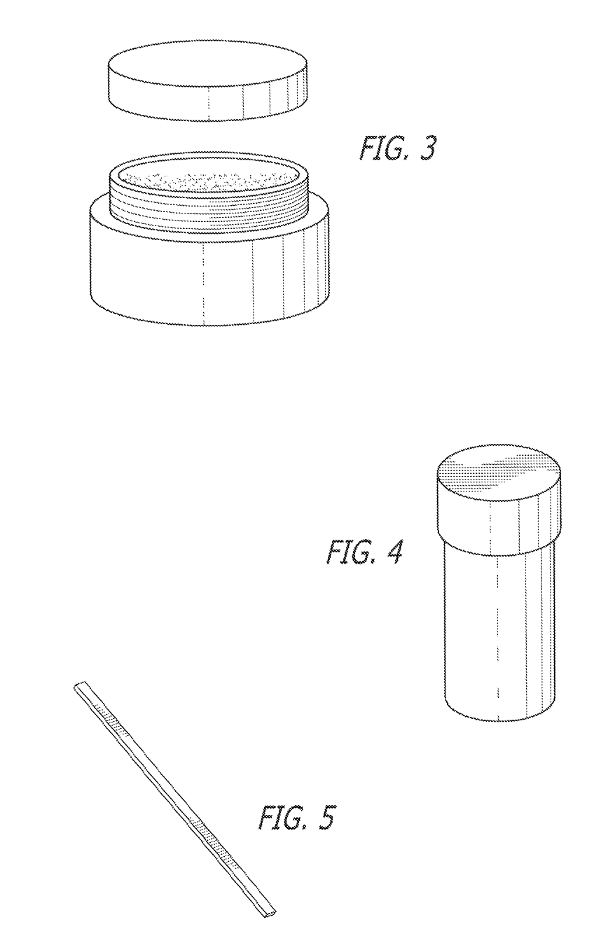

### DETAILED DESCRIPTION OF THE INVENTION

Provided are kits of a base composition and an Active Pharmaceutical Ingredient (API) in separate containers, which are mixed together before administration by a medical professional.

The base composition can be in the same kit with one of the following active ingredients: baclofen, amitriptyline, amitriptyline baclofen, cyclobenzaprine HCl, ibuprofen, lidocaine HCl, lidocaine HCl, naproxen, tramadol, and naproxen. Additional Active Pharmaceutical Ingredients can include: ketoprofen, diclofenac, phenylbutazone, mefenamic acid, flubiprofen, piroxicam guaifenasin, prilocaine, bupivicaine, tetracaine, nifedipine, verapamil, orphenadrine, imipramine, ketamine, gabapentin, carbamazepine, menthol, capsaicin, clonidine, dexamethasone, dextromethorphan, testosterone, progesterone, and estrogens.

The API and the base composition, preferably in the form of a cream, can have the following weight and strength:

| | Kit Total | API | Base | API |
|---|---|---|---|---|
| API | Weight | Weight | Weight | Strength |
| Ketoprofen | 2-260 g | 1-20 g | 1-240 g | 1-20% USP |
| | or 41-140 g | | or 40-120 g | |
| Diclofenac | 2-260 g | 1-20 g | 1-240 g | 1-20% USP |
| | or 41-140 g | | or 40-120 g | |
| phenylbutazone, | 2-260 g | 1-20 g | 1-240 g | 1-20% USP |
| | or 41-140 g | | or 40-120 g | |
| Mefenamic acid | 2-260 g | 1-20 g | 1-240 g | 1-20% USP |
| | or 41-140 g | | or 40-120 g | |
| Flubiprofen | 2-260 g | 1-20 g | 1-240 g | 1-20% USP |
| | or 41-140 g | | or 40-120 g | |
| Piroxicam | 2-260 g | 1-20 g | 1-240 g | 1-20% USP |
| | or 41-140 g | | or 40-120 g | |
| Guaifenasin | 2-260 g | 1-20 g | 1-240 g | 1-20% USP |
| | or 41-140 g | | or 40-120 g | |
| Prilocaine | 2-260 g | 1-20 g | 1-240 g | 1-20% USP |
| | or 41-140 g | | or 40-120 g | |
| Bupivicaine | 2-260 g | 1-20 g | 1-240 g | 1-20% USP |
| | or 41-140 g | | or 40-120 g | |
| Tetracaine | 2-260 g | 1-20 g | 1-240 g | 1-20% USP |
| | or 41-140 g | | or 40-120 g | |
| nifedipine | 2-260 g | 1-20 g | 1-240 g | 1-20% USP |
| | or 41-140 g | | or 40-120 g | |
| verapamil, | 2-260 g | 1-20 g | 1-240 g | 1-20% USP |
| | or 41-140 g | | or 40-120 g | |
| orphenadrine | 2-260 g | 1-20 g | 1-240 g | 1-20% USP |
| | or 41-140 g | | or 40-120 g | |
| Imipramine | 2-260 g | 1-20 g | 1-240 g | 1-20% USP |
| | or 41-140 g | | or 40-120 g | |
| ketamine | 2-260 g | 1-20 g | 1-240 g | 1-20% USP |
| | or 41-140 g | | or 40-120 g | |
| gabapentin | 2-260 g | 1-20 g | 1-240 g | 1-20% USP |
| | or 41-140 g | | or 40-120 g | |
| carbamazepine | 2-260 g | 1-20 g | 1-240 g | 1-20% USP |
| | or 41-140 g | | or 40-120 g | |
| menthol | 2-260 g | 1-20 g | 1-240 g | 1-20% USP |
| | or 41-140 g | | or 40-120 g | |
| capsaicin | 2-260 g | 1-20 g | 1-240 g | 1-20% USP |
| | or 41-140 g | | or 40-120 g | |
| clonidine | 2-260 g | 1-20 g | 1-240 g | 1-20% USP |
| | or 41-140 g | | or 40-120 g | |
| dexamethasone, | 2-260 g | 1-20 g | 1-240 g | 1-20% USP |
| | or 41-140 g | | or 40-120 g | |
| dextromethorphan | 2-260 g | 1-20 g | 1-240 g | 1-20% USP |
| | or 41-140 g | | or 40-120 g | |
| testosterone | 2-260 g | 1-20 g | 1-240 g | 1-20% USP |
| | or 41-140 g | | or 40-120 g | |
| progesterone | 2-260 g | 1-20 g | 1-240 g | 1-20% USP |
| | or 41-140 g | | or 40-120 g | |
| estrogens | 2-260 g | 1-20 g | 1-240 g | 1-20% USP |
| | or 41-140 g | | or 40-120 g | |
| amitriptyline | 2-260 g | 1-20 g | 1-240 g | 1-20% USP |
| | or 41-140 g | | or 40-120 g | |
| baclofen | 2-260 g | 1-20 g | 1-240 g | 1-20% USP |
| | or 41-140 g | | or 40-120 g | |
| cyclobenzaprine | 2-260 g | 1-20 g | 1-240 g | 1-20% USP |
| HCl | or 41-140 g | | or 40-120 g | |
| ibuprofen | 2-260 g | 1-20 g | 1-240 g | 1-20% USP |
| | or 41-140 g | | or 40-120 g | |
| lidocaine HCl | 2-260 g | 1-20 g | 1-240 g | 1-20% USP |
| | or 41-140 g | | or 40-120 g | |
| naproxen | 2-260 g | 1-20 g | 1-240 g | 1-20% USP |
| | or 41-140 g | | or 40-120 g | |
| tramadol | 2-260 g | 1-20 g | 1-240 g | 1-20% USP |
| | or 41-140 g | | or 40-120 g | |

Each kit can contain one container of a base composition, one container of the API, one stirrer, and one package insert. The kit can be used by adding a first portion of the API, such as in powder form, to the container having the base composition to obtain a cream containing the API, stirring the cream, and then adding a second portion of the API to the cream, and stirring until obtaining a homogenous mixture. The first and the second portion together make up 100% of the API. It is believed that the base composition forms liposomes or other vesicles containing the API.

The kit can comprise a container, preferably in the form of a box, in which the above mentioned components are placed. The box can be a rectangular cube with a lid on top for accessing the box.

The base composition can be in the form of a cream and can comprise phospholipids which form a liposomal delivery system containing the API after the base composition is mixed with the API. The obtained pharmaceutical composition in the form of a cream is suitable for applying to the skin for transdermal delivery of the API.

Processes for preparations of liposomes are disclosed in J. Colloid Sci. Biotechnol. 2012, vol. 1, No. 2, which is incorporated herein by reference for its disclosure of processes to prepare liposomes.

The liposome technology can protect and deliver drugs through skin to specific targets in the body. This approach is to greatly reduce the toxicity of many drug compounds that are confined to the carrier and not in the general circulation. The main challenges were the lack of a suitable method to produce liposomal systems with the desired properties and the proper degradation in the circulation of blood fluids.

The liposome technology allows to improve drug delivery as well as performance. Encapsulating Phospholipids have the capability to further reduce toxic concentrations in human organs in pharmaceutical applications. The entrapment has the ability to increase the efficacy and prolong the action of drugs applied topically. This is due to the affinity of the Phospholipid molecular membranes to human cell membranes. This helps to reduce the frequency of administration and risk of over dosage.

The Phospholipid can enhance skin penetration through the preparation of microemulsions. Such systems consist of certain finite ratios of water, oil, and amphiphilic compounds. The resulting compounds are optically transparent, thermodynamically stable, and both hydrophobic and hydrophilic liquids. Microemulsions can be either oil continuous, water continuous, or both. The main difference between macroemulsions and microemulsions lies in the size of the particles of the dispersed phase. Microemulsions are generally about 1/100th the size of macroemulsions.

Penetration enhancement from microemulsions is primarily due to an increase in drug concentration which provides a large concentration gradient from the vehicle to the skin. Furthermore the resulting compounds interact with the rigid lipid bilayer structure and act as a chemical enhancer. The transdermal delivery system can deliver high molecular weight pharmaceuticals and cosmetic agents to skin cells.

Typical liposome has the property of certain amphiphilic molecules forming aggregates in aqueous medium. These molecules are composed of polar and non-polar regions that constitute the hydrophilic head and hydrophobic tail of surfactants. In water, the lipophilic components tend to protect themselves by forming aggregates where hydrophobic components have the minimal contact with water. But this only occurs when certain critical concentration and conditions are met.

Due to the bilayer structure and hydrocarbon chains, the molecules are typically given an overall tubular shape that is more suitable for aggregation. At low concentration in water the stacked bilayers divide into smaller fragments which rearrange to form more stable, tighter, and closed-loop vesicles. This overall mechanism yields liposomes and their sizes vary from 20 nm to several micrometers.

When the sizes are small, their population is relatively homogeneous. Each vesicle is made from one bilayer and the entrapped volume varies, depending on the cube of their radius. The aqueous volume therefore is rapidly reduced when the vesicles becomes smaller, leading to low entrapment efficiency. In addition, the presence of a single bilayer renders them particularly porous to water-soluble molecules.

The most common natural molecules able to form lamellar structures are phospholipids. One of the primary interests in liposomes is due to its ability to entrap both water-soluble and oil-soluble compounds. Such ability allows both hydrophilic and lipophilic ingredients to be carried into vesicular structures which are then suspended in an aqueous medium.

Liposomes have the unique advantage of internalizing water-soluble ingredients inside microscopic particles and can delay the release of the encapsulated material. The entrapped material slowly diffuses out from the loop as the concentration in the external phase decreases. This feature is primarily used for sustained or prolonged actions. Some encapsulated substances can also be protected from the external environment, thus prolonging the half-life of the active ingredients.

The primary transdermal mechanism is the work of the Phospholipid molecules within the compounding systems. Typically the diameter of liposomes formed by the phospholipid is 50-350 nm. The phospholipid content in traditional sense is 0.5-5.0% w/v (mg/ml) Concentration of Phospholipid is in the range of 80-150 mg/L with a purity of 95% or greater. The physical characteristics of phospholipid is a transparent, yellowish gel and the system can be micro-dispersible. Phospholipids can be derived from an enzymatic processes or a non-enzymatic process.

Phospholipids have the ability to encapsulate both water-soluble and oil-soluble compounds within a structure which is biologically compatible with the skin. The Phospholipid molecules can be modified to improve stability and delay reaction time.

The primary reason for the absence or slow commercial applications of such vesicles is the difficulty in producing large volumes of material and secondly their low encapsulation efficiencies. Most drug or dermatological preparations contain active ingredients that can only act after they penetrate at least the outermost layer of the skin. Then the efficacy of topically applied actives is often suboptimal because the transport into the skin is slow due to the resistance of the outermost layer of the skin, the stratum corneum. In the absence of the horny layer, virtually all small water-soluble non-electrolytes can diffuse into the systemic circulation a thousand times more rapidly.

Controlling the pH, pressure, solvent percentages, temperature, and swelling of molecules are all critically important factors in phospholipid production. Other technical factors important in phospholipid production include the physical presence of the vesicles, sizes and lamellarity, and the entrapped volume. Controlling the size is particularly important to maintain target stability, storage, loading, and transdermal penetration.

Skin is the largest human organ and consists of three functional layers—epidermis, dermis, and subcutis. One primary function of the skin is to protect the organism from water loss and mechanical, chemical, microbial, and physical environments. The protective properties are provided by the outermost layer of the skin, the epidermis. Although its thickness measures on average only 0.1 mm within the range of 0.02-5 mm, it is specially structured to perform this task well. Out of the five layers of the epidermis, it is mainly the uppermost layer, the horny layer or stratum corneum, which forms the permeability barrier. The stratum corneum consists of horny skin cells known as corneocytes which are connected via desmosomes which is protein-rich appendages of the cell membrane. The corneocytes are embedded in a lipid matrix. The corneocytes of hydrated keratin can be illustrated as the bricks and the epidermal lipids fill the space between the dead cells like mortar.

The epidermal lipids comprise 10 to 30% of the total volume of the stratum corneum. The major components are ceramides, fatty acids, cholesterol, and cholesterol esters. The lipids are organized as multiple lipid sandwiches of regions of semi-crystalline gel and liquid crystals domains.

There are possible pathways for a penetrant to cross the skin barrier. A molecule may use two diffusional routes to penetrate normal intact human skin—the appendageal route and the transepidermal route. The appendageal route comprises transport via the sweat glands and the hair follicles with their associated sebaceous glands. These routes circumvent penetration through the stratum corneum. Although these routes offer high permeability, they are of minor importance because of their relatively small area, approximately 0.1% of the total skin area. The appendageal route seems to be important for ions and large polar molecules which are not readily permeable via the stratum corneum.

Transepidermal transport occurs when molecules cross the intact horny layer. Two potential micro-routes of entry exist—the transcellular or intracellular and the intercellular pathways. The preferred decision on the penetrant pathway is made by the partition coefficient. Hydrophilic drugs partition preferentially into the intracellular domains, whereas lipophilic permeants traverse the stratum corneum via the intercellular route. Most molecules pass the stratum corneum by both routes. However for most drug compounds, the tortuous intercellular pathway is the most probable route of penetration.

The skin provides a protective barrier against foreign materials and infection. In human this is accomplished by forming a highly insoluble protein and lipid structure on the surface of the corneocytes termed the cornified envelope. The cornified envelope is composed of polar lipids, such as ceramides, sterols, and fatty acids, and a complicated network of cross-linked proteins. However, the cytoplasm of stratum corneum cells remains polar and aqueous. The cornified envelope extremely thin at 10 microns but provides a substantial barrier. Because of the accessibility and large area of the skin, it can be a promising route for the administration of drugs, whether dermal, regional, or systemic effects are desired.

A topical route of drug administration is sometimes desirable because the risks and inconvenience of parenteral treatment can be avoided. The variable absorption and metabolism associated with oral treatment can also be circumvented. Another benefit is that drug administration can be continuous, thereby permitting the use of pharmacologically active agents with short biological half-lives. The gastrointestinal irritation associated with many compounds can be avoided and cutaneous manifestations of diseases can be treated more effectively than by systemic approaches.

Most transdermal delivery systems achieve epidermal penetration by using a skin penetration enhancing vehicle. While many of the skin enhancers in the literature enhance transdermal absorption, several possess certain drawbacks in that some are regarded as toxic; some irritate the skin; some on prolonged use have a thinning effect on the skin; some change the intactness of the skin structure, resulting in a change in the diffusability of the drug; and all are incapable of delivering high molecular weight pharmaceuticals and cosmetic agents.

Phospholipids offer safe and effective transdermal delivery systems that can administer a wide-range of pharmaceuticals and cosmetic agents to skin cells. Phospholipid and its transdermal delivery systems can be incorporated to administer pharmaceuticals and cosmetic agents of various molecular weights.

There are several systems that can effectively delivery active transdermally. These can administer an effective amount of is pharmaceutical or cosmetic agent to the human body. This system can be used to administer low and/or high molecular weight pharmaceuticals and cosmetic agents in amounts that are therapeutically, prophylactically, or cosmetically beneficial.

In some embodiments, a transdermal delivery system comprises a combination of the delivery agents. The second component of a transdermal delivery system is a penetration enhancer. Desirable penetration enhancers comprise both hydrophobic and hydrophilic components. The hydrophobic component includes one or more proprietary compounds.

The amount of delivery agent that is incorporated into the penetration enhancer depends on the compound, desired dosage, and application while maintaining strict ratios among the key components. The amount of delivery agent in a particular formulation can be expressed in terms of percentage by weight, percentage by volume, or concentration. Additionally, some delivery agents can be solubilized in the aqueous adjuvant prior to mixing with the penetration enhancer.

A transdermal delivery system has three components—a delivery agent, a penetration enhancer, and an aqueous adjuvant. One component of the transdermal delivery system of can be a molecule or a mixture of molecules that are delivered to the body using an embodiment of a transdermal delivery system. A delivery agent that can be administered to the body using an embodiment of the invention can include, for example, a protein, a sugar, a nucleic acid, a chemical, or a lipid.

Using phospholipid allows for adjusting the dosage and administration according to the target levels of the active moiety. Additional factors that may be taken into account include the severity of the disease state, age, weight and gender of the patient; diet, time and frequency of administration, drug combinations, reaction sensitivities, and tolerance/response to therapy.

Short acting compositions can be administered frequently whereas long acting pharmaceutical compositions can be varied widely. The frequency and dosage of administration is also highly dependent on half-life and clearance rate of the particular formulation. Routes of administration of the delivery systems of phospholipid are primarily topical, although the core technology can easily be incorporated into other potential embodiments to cells.

Compositions of the base composition suitable for topical application include, but are not limited to, physiologically acceptable ointments, creams, rinses, and gels.

A collection of systems that has the ability to squeeze the molecules through pores less than one-tenth of their diameter can also be used.

In one embodiment, the base composition is made from the following ingredients: water, carthamus tinctorius seed oil, aloe barbadensis leaf extract, helianthus annus seed oil, olea europaea fruit oil, butyrospermum parkii butter, beeswax, cetearyl alcohol, glyceryl stearate, perilla ocymoides seed oil, isopropyl palmitate, lysolecithin, hydrogenated lecithin, perilla ocymoides leaf extract, isopropyl myristate, squalane, punica granatum extract, tocopheryl acetate, cholocalciferol, cetyl alcohol, retinyl palmitate, vitis vinifera seed extract, wheat amino acids, triticum vulgare germ extract, cenothera biennis oil, xaltham gum, and phenoxyethanol. The base composition can have anywhere from 15 (e.g. 15, 16, 17, 18, 19, 20, 21, 22, 23, 24, 25, 26, etc.) to all of these ingredients. In one embodiment, the base composition is made from anywhere from 5 (e.g. 6, 7, 8, 9, 10, 11, etc.) of these ingredients: carthamus tinctorius seed oil, helianthus annus seed oil, olea europaea fruit oil, butyrospermum parkii butter, perilla ocymoides seed oil, cenothera biennis oil, squalane, lysolecithin, hydrogenated lecithin, isopropyl palmitate, isopropyl myristate, tocopheryl acetate, retinyl palmitate. In one embodiment, the base composition is made (either alone or in combination with the above ingredients) from anywhere from 3 (e.g. 4, 5, 6) of these extracts: aloe barbadensis leaf extract, perilla ocymoides leaf extract, perilla ocymoides leaf extract, punica granatum extract, vitis vinifera seed extract, triticum vulgare germ extract.

### EXAMPLES

### Example 1

The following kits were made with the following base composition in the form of a cream and an API placed in its own individual container in the form of a powder. Each of the containers was placed in a box having a lid along with a stirrer and an instruction sheet.

| Kit | | | | Base Cream | | | | |
|---|---|---|---|---|---|---|---|---|
| # | API | Weight | | Composition | | API | | API Strength |
| | amitriptyline | 60 | g | 58.8 | g | 1.2 | g | 2% USP |
| 1 | amitriptyline | 120 | g | 117.6 | g | 2.4 | g | 2% USP |
| 2 | baclofen | 60 | g | 59.4 | g | 0.6 | g | 1% USP |
| 3 | baclofen | 120 | g | 118.8 | g | 1.2 | g | 1% USP |
| 4 | cyclobenzaprine | 120 | g | 117.6 | g | 2.4 | g | 2% USP |
| | HCl | | | | | | | |
| 5 | ibuprofen | 120 | g, | 108 | g | 12 | g | 10% USP |
| 6 | lidocaine HCl | 60 | g | 54 | g | 6 | g | 10% USP |
| 7 | lidocaine HCl | 60 | g | 57 | g | 3 | g | 5% USP |
| 8 | lidocaine HCl | 120 | g | 108 | g | 12 | g | 10% USP |
| 8 | lidocaine HCl | 120 | g | 114 | g | 6 | g | 5% USP |
| 9 | naproxen | 60 | g | 54 | g | 6 | g | 10% USP |
| 10 | naproxen | 120 | g | 108 | g | 12 | g | 10% USP |
| 11 | tramadol | 60 | g | 57 | g | 3 | g | 5% USP |

| | | | | | | |
|---|---|---|---|---|---|---|
| 12 | tramadol | 120 | g | 114 | 6 | 5% USP |

The base composition was made from the following ingredients (Available under the name of Microderm™ base from Enovachem Manufacturing (Torrance, Calif.)):

water
carthamus tinctorius seed oil
aloe barbadensis leaf extract
helianthus annus seed oil
Olea europaea fruit oil
butyrospermum parkii butter
beeswax
cetearyl alcohol
glyceryl stearate
perila ocymoides seed oil
isopropyl palmitate
lysolecithin
hydrogenated lecithin
perilla ocymoides leaf extract
isopropyl myristate
squalane
punica granatum extract
tocopheryl acetate
cholecalciferol
cetyl alcohol
retinyl palmitate
vitis vinifera seed extract
wheat amino acids
triticum vulgare germ extract
cenothera biennis oil
xaltham gum
phenoxyethanol

Each kit contained one container of a base composition, one container of the API in powder form, one stirrer, and one product package insert stating:

1. Slowly add about half of the API powder into the container containing the base;
2. Gently stir cream with stirrer for 1-2 minutes
3. Add the remaining API powder into the container containing the cream and gently stir with stirrer for an additional 2-3 minutes until the final product is homogenous in appearance.

### Example 2

### Prophetic Example for Preparation of a Base Composition on the Form of a Cream

### Name:

General base cream

### Dosage Form:

Emulsion/cream

### Route of Administration:

Topical

### Formula:

| | | | Therapeutic |
|---|---|---|---|
| Ingredient | Quantity | | Activity |
| Cetyl alcohol | 3.56% | w/w | |
| Stearic acid | 4.8% | w/w | |
| Olive oil | 5.78% | w/w | solvent |
| Jojoba oil | 0.5% | w/w | solvent |
| Tea tree oil | 0.51% | w/w | solvent |
| Polysorbitan | 1.78% | w/w | surfactant |
| monooleate | | | |
| (Span 60) | | | |
| Polysorbitan | 0.75% | w/w | surfactant |
| monostearate | | | |
| (Tween 80) | | | |
| Double | 50 | mL | solvent |
| distilled water | | | |

### Method of Preparation:

The oil components (all components except water) are mixed with a magnetic stirrer@200 rpm and 70° C. Water, heated to 70° C. is then added at a rate of 30 mL/min to the oil phase with stirring maintained at 250 rpm.

### Literature Information:

C. D. Kaur and S. Saraf “Topical vesicular formulations of Curcuma longa extract on recuperating the ultraviolet radiation-damaged skin” Journal of Cosmetic Dermatology, (2011) 10, 260-265.

### Example 3

### Prophetic Example for Preparation of a Base Composition on the Form of a Cream

Pluronic Lethecin Organogel (PLO) cream is prepared. The oil phase is prepared by mixing lecithin and isopropyl palmitate and allowing the mixture to stand overnight to ensure complete dissolution. The aqueous phase is prepared by adding Pluronic F127 to ice cold water, placing the mixture in a refrigerator and agitating periodically to ensure complete dissolution. To prepare PLO, the oil phase is then mixed with the aqueous phase using a high-shear mixing method.

### Literature Information:

Hospital Pharmacist, Vol. 12, 267.

What is claimed is:

1. A kit comprising:
a) a first container of an active pharmaceutical ingredient (API);
b) a second container of a base composition suitable for transdermal delivery for mixing with the API; and
c) a third container;
wherein the first container and the second container are placed inside a third container.

2. The kit of claim 1, wherein the base composition in form of an ointment, a cream, a rinse, or a gel.

3. The kit of claim 1, wherein the base composition in form of a cream.

4. The kit of claim 1, wherein the API is selected from the group consisting of: amitriptyline, baclofen, cyclobenzaprine HCl, ibuprofen, lidocaine HCl, naproxen, and tramadol.

5. The kit of claim 1, wherein the API is selected from the group consisting of: ketoprofen, diclofenac, phenylbutazone, mefenamic acid, flubiprofen, piroxicam, guaifenasin, prilocaine, bupivicaine, tetracaine, nifedipine, verapamil, orphenadrine, imipramine, ketamine, gabapentin carbamazepine, menthol, capsaicin, clonidine, dexamethasone, dextromethorphan, testosterone, progesterone, and estrogens.

6. The kit of claim 1, further comprising a stirrer placed inside the third container.

7. The kit of claim 1, further comprising an instruction sheet placed inside the third container.

8. The kit of claim 1, wherein the third container is a box with a lid.

9. The kit of claim 1 wherein the API is amitriptyline.

10. The kit of claim 1, wherein the API is baclofen.

11. The kit of claim 1, wherein the API is cyclobenzaprine HCl.

12. The kit of claim 1, wherein the API is ibuprofen.

13. The kit of claim 1, wherein the API is lidocaine HCl.

14. The kit of claim 1, wherein the API is naproxen.

15. The kit of claim 1, wherein the API is tramadol.

16. The kit of claim 1, wherein the kit comprises:
a) 1-20 grams of the API, with the API having a strength of 1-20% USP (United States Pharmacopeia); and
b) 1-240 grams of the base composition.

17. The kit of claim 1, wherein the kit comprises
a) 1-20 grams of the API, with the API having a strength of 1-20% USP (United States Pharmacopeia); and
b) 40-120 grams of the base composition.

18. The kit of claim 1, wherein weight of the API and the base composition in range of about 40 grams to about 140 grams.

19. The kit of claim 1, wherein weight of the API and the base composition in range of about 60 grams to about 120 grams.

20. The kit of claim 1, wherein weight of the API is 1 gram to about 20 grams.

21. The kit of claim 1, wherein strength of the API is about 1% to about 10% USP (United States Pharmacopeia).

22. The kit of claim 1, wherein weight of the API and the base composition in the range of about 40 grams to about 140 grams, with the API being present in amount of 1 gram to about 20 grams, with the rest being weight of the base composition.

23. The kit of claim 1, wherein the API is amitriptyline, weight of the API and the base composition is about 60 grams, with weight of the API being 1.2 grams, and wherein the API has strength of 2% USP (United States Pharmacopeia).

24. The kit of claim 1, wherein the API is amitriptyline, weight of the API and the base composition is about 120 grams, with weight of the API being 2.4 grams, and wherein the API has strength of 2% USP (United States Pharmacopeia).

25. The kit of claim 1, wherein the API is baclofen, weight of the API and the base composition is about 60 grams, with weight of the API being 0.6 grams, and wherein the API has strength of 1% USP (United States Pharmacopeia).

26. The kit of claim 1, wherein the API is baclofen, weight of the API and the base composition is about 120 grams, with weight of the API being 1.2 grams, and wherein the API has strength of 1% USP (United States Pharmacopeia).

27. The kit of claim 1, wherein the API is cyclobenzaprine HCl, weight of the API and the base composition is about 120 grams, with weight of the API being 2.4 grams, and wherein the API has a strength of 2% USP (United States Pharmacopeia).

28. The kit of claim 1, wherein the API is ibuprofen, weight of the API and the base composition is about 120 grams, with weight of the API being 12 grams, and wherein the API has strength of 10% USP (United States Pharmacopeia).

29. The kit of claim 1, wherein the API is lidocaine HCl, weight of the API and the base composition is about 60 grams, with weight of the API being 6 grams, and wherein the API has strength of 10% USP (United States Pharmacopeia).

30. The kit of claim 1, wherein the API is lidocaine HCl, weight of the API and the base composition is about 60 grams, with the weight of the API being 3 grams, and wherein the API has strength of 5% USP (United States Pharmacopeia).

31. The kit of claim 1, wherein the API is lidocaine HCl, weight of the API and the base composition is about 120 grams, with the weight of the API being 12 grams, and wherein the API has strength of 10% USP (United States Pharmacopeia).

32. The kit of claim 1, wherein the API is lidocaine HCl, weight of the API and the base composition is about 120 grams, with the weight of the API being 6 grams, and wherein the API has strength of 5% USP (United States Pharmacopeia).

33. The kit of claim 1, wherein the API is naproxen, weight of the API and the base composition is about 60 grams, with the weight of the API being 6 grams, and wherein the API has strength of 10% USP (United States Pharmacopeia).

34. The kit of claim 1, wherein the API is naproxen, weight of the API and the base composition is about 120 grams, with the weight of the API being 12 grams, and wherein the API has strength of 10% USP (United States Pharmacopeia).

35. The kit of claim 1, wherein the API is tramadol, weight of the API and the base composition is about 60 grams, with the weight of the API being 3 grams, and wherein the API has strength of 5% USP (United States Pharmacopeia).

36. The kit of claim 1, wherein the API is tramadol, weight of the API and the base composition is about 120 grams, with the weight of the API being 6 grams, and wherein the API has strength of 5% USP (United States Pharmacopeia).

37. A method of transdermally administering an Active Pharmaceutical Ingredient (API) comprising:
a) removing a first container of a base composition from a third container;
b) removing a second container of an active pharmaceutical ingredient (API) from the third container;
c) opening the first container;
d) opening the second container;
e) mixing the active pharmaceutical ingredient (API) and the base composition to obtain a pharmaceutical composition for transdermal delivery; and
f) applying the pharmaceutical composition to skin.
